(19) United States
(12) Plant Patent Application Publication
(10) Pub. No.: US 2005/0198721 P1
(43) Pub. Date: Sep. 8, 2005

(54) VERBENA X HYBRIDA PLANT NAMED 'VERENA'

(50) VERBENA X HYBRIDA; VERENA

(76) Inventor: Henricus Stemkens, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/795,614

(22) Filed: Mar. 8, 2004

(51) Int. Cl. A01H 5/00

(52) U.S. Cl. PLT/308

(57) ABSTRACT

A new and distinct variety of Verbena plant named ‘Verena’ particularly distinguished by its deeppink flower, incised leaves, early flowering and a spreading habit that is first sermi-erect and later spreading.

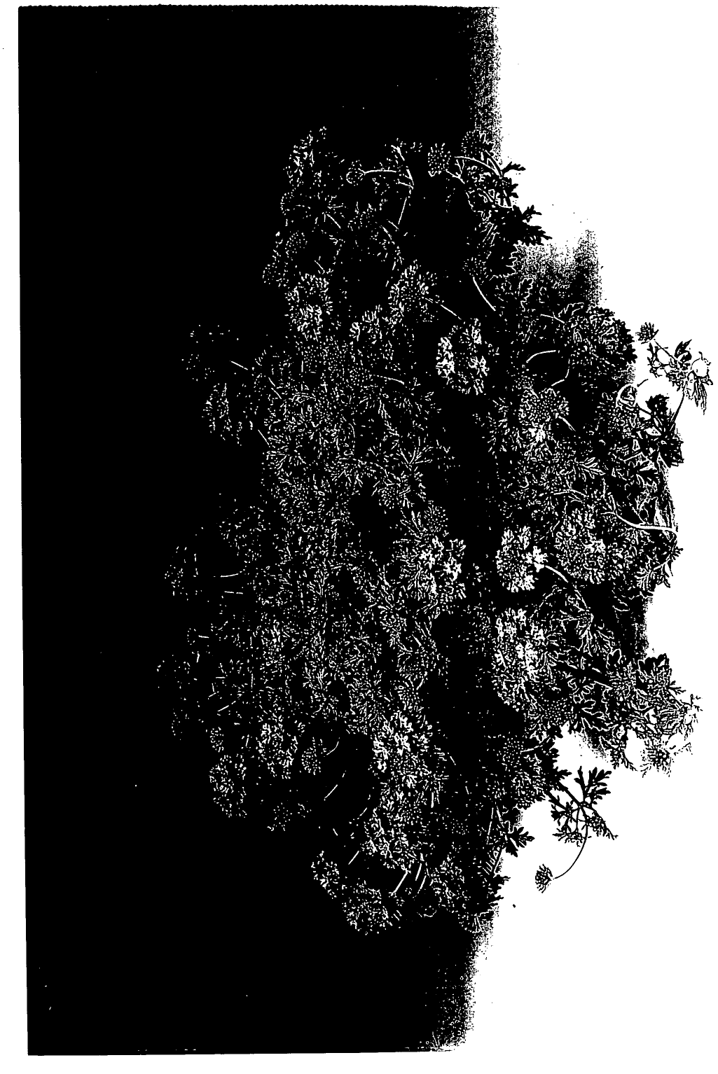

### LATIN NAME OF THE GENUS AND SPECIES OF THE PLANT CLAIMED

Verbena×hybrida

### VARIETAL DENOMINATION

‘Verena’

### BACKGROUND OF THE NEW PLANT

The present invention comprises a new distinct cultivar of Verbena, botanically known as Verbena×hybrida. The new cultivar is propagated from cuttings resulting from the cross of ‘W720’ and ‘T593’. ‘W720’ is a rose flowering Verbena having a spreading habit. ‘W720’ is not commercially available and is not known by any synonyms. ‘T593’ is a scarlet flowering Verbena having a semi-erect habit. ‘T593’ is not commercially available and is not known by any synonyms. Neither ‘W720’ or ‘T593’ has been patented. As a result of this cross the present cultivar was created in 1999 in Enkhuizen, Netherlands and has been repeatedly asexually reproduced by cuttings in Enkhuizen, Netherlands and Sarrians, France over a three year period. It has been found to retain its distinctive characteristics through successive propagations, and this novelty appears to be firmly fixed.

This new Verbena plant is an annual in most climatical zones in the US, only in zones 9 and 10 it is aperennial plant.

### DDESCRIPTION OF THE DRAWING

This new Verbena plant is illustrated by the accompanying photographic drawing which shows blooms, buds and foliage of the plant in full colour, the colour shown being as true as can be reasonably obtained by conventional photographic procedures.

### DESCRIPTION OF THE NEW CULTIVAR

The following detailed descriptions set forth the distinctive characteristics of this new Verbena. The data which defines these characteristics were collected from asexual reproductions carried out in Enkhuizen, Netherlands. The plant history was taken on 14 weeks old plants, blossomed under natural light in a greenhouse. Colour readings were taken in the greenhouse under ambient light. Colour references are primarily to the RHS Colour Chart of The Royal Horticultural Society of London.

### Specification of Differences with Parents and Most Resembling Variety

| | Character | | | |
|---|---|---|---|---|
| | | | | ‘Temari |
| | ‘Verena’ | ‘W720’ | ‘T593’ | coral rose’ |
| Flowercolor | deeppink | rose | scarlet | coral rose |
| Earliness | early | very early | late | late |
| Seedset | no | no | much | abundant |
| Leave | double | double | no | no |
| incision | incised | incised | incisions | incisions |

### The Plant

Classification—botanical: Verbena×hybrida.
Parentage—female parent: A seedling named ‘W720’ is one of our seedlings from our W-generation of plants bred in 1994. — Pollen parent A seedling named ‘T593’ is one of our seedlings from our T-generation of plants bred in 1991.
Growth habit: Semi-erect later spreading.
Plant height: 18-26 cm.
Spreading area of plant: 32-60 cm.
Growth rate: Hanging and vigorous.
Strength: Resistant to hot and cool weather.
Branching character: Freely branching and lateral branching at every node.
Blooming period: From April till November.

### The Stem

Diameter: 2 to 2.5 mm.
Shape: Tetragonal.
Anthocyan pigmentation: Present.
Colour: 141C.
Length of internode: 25 to 35 mm, depending on the light where the plant is propagated.
Pubescence: Pubescence is present.

### The Foliage

Phyllotaxis: Opposite.
Shape of blade: Broadly ovate.
Texture upper side: Smooth lower side: Smooth.
Venation: Pinnate.
Leaf margin: Laciniate.
Leaf base: Hastate.
Leaf apex: Acute.
Length: 16 to 25 mm.
Width: 14 to 22 mm.
Depth of incision: 8 to 10 mm.
Number of incisions: 2 to 5 per leaf.
Colour—upper side: 141B — Lower side 138B.
Pubescence: Some pubescence is present.
Length of leaf stem: 8 to 12 mm.
Petiole surface structure: Slightly pubescent.

### The Bud

Peduncle length: 20 to 30 mm, depending on season.
Size—Diameter: 2 mm — Length: 8 to 10 mm.
Shape: Elongated and ovate.
Colour: 137C.
Sepals—colour: 138C — Form: Upright — Number 5, fused — Size 5 mm — Shape Elongated.

### The Flower

Facing direction: Upward.
Outward curvature of petal: Slightly curved.
Diameter: 14 to 18 mm.
Height: 14 to 16 mm.
Borne: In a cluster.
Form: Salverform; sessile on terminal spikes.
Cluster: Spike.
Colour: 37A.
Eye: A very small (1 mm) greenish eye (155B) is present Typically three out of the five petals exhibit this greenish coloration.
Overlapping of petals: Separate.
No. of petals: Gamopetalous, Slobed.
Shape of the petals: Each petal is heart shaped at the apex and grown together at the base.
Petal margin: Entire.
Petal surface texture: Smooth.
Size of the petal—length: 4 to 6 mm — Width: 5 to 8 mm.
Spike—length: 32 to 40 mm — Diameter 35 to 45 mm.
Calyx length: 8 to 10 mm.
Anthocyan pigmentation of calyx limb: Absent.
Color of the calyx: 137A.
No. of flowers per spike: 25 to 45.
Fragrance: A very light rosy fragrance.
Lastingness of the bloom: New florets continue to open in one spike over a period of 14 days.

### Reproductive Organs

Androecium:
   Stamens quantity.—4.
   Anther shape.—ovoid.
   Anther length.—1 mm.
   Anther color.—144C.
   Pollen amount.—no pollen.
Gynoecium:
   Pistils quantity.—1.
   Pistil length.—1.8 to 2.2 cm.
   Stigma shape.—bi-lobed.
   Stigma color.—144C.
   Style length.—1.6 cm.
   Style color.—144D.
   Ovary color.—144C No seedset is observed.

### Roots

Type of roots: Fibrous. Roots start to grow on every part of the stem that contacts the soil, so not only at the nodes.

### Physiological and Ecological Characteristics

Good tolerance to heat and cold. Strong resistance to pests and diseases, particularly powdery mildew.

1. A new and distinct variety of Verbena plant, substantially as herein illustrated and described, characterized particularly as to novelty by deeppink flowers, which appear earlier on the plant, by incised darkgreen leaves and a growing habit that is first semi- erect and later spreading.
